(19) United States
(12) Patent Application Publication
(10) Pub. No.: US 2026/0151153 A1
(43) Pub. Date: Jun. 4, 2026

(54) COUPLING DEVICE AND HANDLE DEVICE FOR A SURGICAL INSTRUMENT, SURGICAL INSTRUMENT AND METHOD FOR ASSEMBLY AND METHOD FOR DISASSEMBLY OF THE SURGICAL INSTRUMENT

(71) Applicant: KARL STORZ SE & Co. KG, Tuttlingen (DE)

(72) Inventors: Janosz SCHNEIDER, Tuttlingen (DE); Daniel KÄRCHER, Tuttlingen (DE); Robin MERZ, Tuttlingen (DE); Sven SCHNEIDER, Tuttlingen (DE); Tobias UNGER, Tuttlingen (DE); Dominik LÄNGLE, Tuttlingen (DE); Judith PÖNISCH, Tuttlingen (DE)

(21) Appl. No.: 19/115,400

(22) PCT Filed: Sep. 29, 2023
(86) PCT No.: PCT/EP2023/077019

§ 371 Date: Mar. 26, 2025

(30) Foreign Application Priority Data
Sep. 29, 2022 (DE) 10 2022 125 213.2

(51) Int. Cl. A61B 17/29; A61B 17/00

(52) U.S. Cl. CPC A61B 17/2909; A61B 2017/0046; A61B 2017/292

(57) ABSTRACT

A coupling device for a surgical instrument, a handle device for it, and corresponding assembly and disassembly methods. The device has a force transmission element defining a longitudinal axis with an offset connection element at its proximal end, and a handle part movable about a pivot axis perpendicular to the longitudinal axis. Having a coupling element with a recess for receiving the connection element and a slider element which cooperates with the coupling element and is configured to cooperate with the movable handle part and to move in the direction of the longitudinal axis. Receiving in a guide receptacle of the slider element such that it can move along a coupling axis, and the recess in the coupling element corresponds to the coupling axis. Having a notch connected to the recess. The coupling element can move along the coupling axis between at least one release position and one engaging position.

### CROSS REFERENCE TO RELATED APPLICATIONS

This application is a United States National Phase Application of International Application PCT/EP2023/077019, filed Sep. 29, 2023, and claims the benefit of priority under 35 U.S.C. § 119 of German Application 10 2022 125 213.2, filed Sep. 29, 2022, the entire contents of which are incorporated herein by reference.

### TECHNICAL FIELD

The invention relates to a coupling device for a surgical instrument and a handle device of a surgical instrument with such a coupling device. Furthermore, the invention relates to a surgical instrument with such a coupling device itself as well as a method for assembling and a method for disassembling the surgical instrument.

### BACKGROUND

It is known from the prior art that medical instruments for endoscopic surgery have a movable tool at the distal end of an elongated shaft, at the proximal end of which an actuating unit such as a handle with a fixed and a movable handle part for actuating the tool is arranged. For this purpose, the surgical instrument has a force transmission element, for which a pull and push rod (abbreviated to pull rod) is often used, and which brings the tool into operative connection with the handle, or the movable handle part. For this purpose, the force transmission element extends axially movably through the elongated shaft and is connected on the distal side to a tool mechanism which transmits the longitudinal axial back and forth movement of the force transmission element to the tool, for example for opening and closing jaw parts of the tool, for example for gripping or cutting. At the proximal end, the force transmission element engages with the movable handle part, by which a movement of the movable handle part toward or away from the fixed handle part is converted into the longitudinal axial back and forth movement of the force transmission element.

Furthermore, it is known that modular instrument systems exist from which such medical instruments can be assembled, for example, from the assemblies handle, shaft and working insert, consisting of force transmission element and tool, which can also be disassembled, cleaned and disinfected and are therefore at least partially reusable. Such a modular surgical instrument system is known, for example, under the name Clickline® Instruments from the catalog “Highlights, Clickline® Instruments, Laparoscopic Hand Instruments, 01/2020” of Karl Storz GmbH & Co., Tuttlingen, Germany. Modular instrument systems also increase functional variability by allowing different handles to be combined with different shafts and/or working inserts. On the one hand, the different handles can differ in terms of the maximum force that can be generated due to different lever ratios, and on the other hand, the force transmission elements can differ in terms of the maximum force that can be transmitted with them. The maximum force that can be transmitted with the particular force transmission element can be adapted to the task of the tool at the distal end and limited by the strength of the force transmission element or its proximal connection to a movable handle part.

The force transmission elements of an instrument system that can be disassembled or the proximal connection of the force transmission elements with a movable handle part can be dimensioned differently depending on the different areas of use and specifications.

For the force-locking connection of such force transmission elements with a movable handle part, EP 2 305 145 B1, which relates to such a medical forceps system capable of being disassembled, describes coupling devices for engagement with a connection element at the proximal end of the force transmission element. The connection elements of the force transmission elements have different cross sections for the different areas of use and specifications. A connection element can be received in a correspondingly designed receiving recess of a coupling element which is connected to a slide which can be actuated by the movable handle part. The first receiving recess can be followed on the proximal side of the coupling element by a further recess with a smaller diameter, which allows the accommodation of a connection element with a correspondingly smaller diameter. For the positive engagement of the connection element in each case, a rotation of the coupling element about the axis of the force transmission element is required.

However, the positive connection of such a spherical connection element of the actuating element in a spherical receiving recess of the coupling element is subject to play. Furthermore, the coupling device is designed for a maximum of two ball sizes, which reduces variability with regard to different tools, so that additional force limiting devices must be provided, especially when using distal tools with a low loading capacity, in order to protect the distal tool from overloading due to excessive actuating force on the handle by the user.

### SUMMARY

Based on this prior art, it is an object of the present invention to provide an improved coupling device for a surgical instrument.

This object is achieved by a coupling device having features according to the invention.

The further objects of providing a correspondingly improved handle device for a surgical instrument and a correspondingly improved surgical instrument are achieved by a handle device having the features and by a surgical instrument having features according to the invention.

A simplified assembly of a surgical instrument and its disassembly are achieved by the methods having features according to the invention.

Further developments and preferred embodiments are set out in this disclosure, including the description, figures and claims.

According to a first embodiment, a coupling device according to the invention is provided for a surgical instrument which has a force transmission element and a handle device with a movable handle part. The terms “proximal” and “distal” are used herein to indicate the location of components in relation to a user operating the surgical instrument. Accordingly, the handle device is connected to a proximal end of the force transmission element, the other—distal—end of which is usually connected to a tool. The coupling device is configured to connect the movable handle part to the proximal end of the force transmission element in order to convert an actuating movement applied to the handle part by user force into a longitudinal movement of the force transmission element. For this purpose, the force transmission element, which defines a longitudinal axis corresponding to the longitudinal axis of the surgical instrument, has an offset connection element at its proximal end. The step is formed by a change in the cross-sectional dimensions of the connection element with respect to the force transmission element perpendicular to the longitudinal axis, wherein the cross-sectional dimensions of the step are smaller than the cross-sectional dimensions of the connection element. The movable handle part of the handle device is movable about a pivot axis which runs perpendicular to the longitudinal axis but does not intersect it. The coupling device for connecting the force transmission element to the movable handle part has a coupling element and a slider element cooperating therewith. A recess for receiving the connection element is formed in the coupling element, and the slider element, which can be moved in the direction of the longitudinal axis, is configured to cooperate with the movable handle part.

According to the invention, a guide receptacle for the coupling element is formed in the slider element along a coupling axis which runs perpendicular to the longitudinal axis and perpendicular to the pivot axis. The coupling element received in the guide receptacle can thus be moved along the coupling axis. The recess in the coupling element, which is provided for receiving the connection element at the proximal end of the force transmission element, runs corresponding to the coupling axis, i.e., along the coupling axis or with a certain deviation from the coupling axis. In this case, an axis of rotation or axis of symmetry of the recess can be identical to the coupling axis; alternatively, the rotation or symmetry axis of the recess can be offset parallel to the coupling axis in a plane defined by the longitudinal and coupling axis, or can run at a predetermined angle to the coupling axis in the plane defined by the longitudinal and coupling axis. Furthermore, the cross section of the recess in the coupling element tapers in the direction of the coupling axis, starting from an opening side of the coupling element which points away from the guide receptacle. The cross section of the recess is thus largest on the opening side of the coupling element and becomes smaller with increasing distance from the opening side, wherein at least the cross-sectional dimension which runs along or parallel to the longitudinal axis decreases. In addition, the coupling element has a notch on the distal side which is connected to the recess. The width of the notch also tapers in a direction parallel to the coupling axis, with the width of the notch being largest at the opening side of the coupling element and becoming smaller with increasing distance from the opening side.

“Cross section” of the recess refers to a surface perpendicular to the coupling axis. The term “width” refers to dimensions in a direction that is perpendicular to the longitudinal axis and perpendicular to the coupling axis, i.e., parallel to the pivot axis.

The coupling element can be positioned in the slider element along the coupling axis in at least one release position in which the connection element can be released from the coupling element and at least one engaging position in which the connection element is connected to the coupling element.

The coupling element is in a release position along the coupling axis when, at a height of the longitudinal axis, the width of the notch is greater than a width of the connection element, so that the connection element cannot engage behind the notch, but can be inserted into and pulled out of the recess through a notch when the force transmission element moves in the direction of the longitudinal axis. The release position of the coupling element is also understood to mean a positioning in which the coupling element is moved away from the longitudinal axis along the coupling axis until the connection element is outside the recess. Since the notch width outside the recess extends virtually around the entire circumference of the recess, in such a case the width of the notch on the opening side of the recess can be smaller than the width of the connection element.

An engaging position along the coupling axis is assumed by the coupling element when, at the height of the longitudinal axis, the cross-sectional dimension of the recess in the direction of the longitudinal axis corresponds to a longitudinal section dimension of the connection element and, in addition, the width of the notch is smaller than the width of the connection element, wherein the width of the notch corresponds at least to a width of the step with which the connection element is offset from the force transmission element. The connection element then engages behind the notch and comes to rest in the recess. The connection element rests against the inner wall with at least one proximal portion opposite the notch and contacts, on both sides, the inner wall portions on the distal side which delimit the notch. In this way, the force transmission element is connected to the coupling device without play in the longitudinal direction. In a surgical instrument, this enables a precise longitudinal movement of the force transmission element through the handle part to actuate a distal tool, the functional reliability of which is thereby improved. Furthermore, the coupling device according to the invention simplifies the disassembly and assembly of a surgical instrument by the coupling element being movable along the coupling axis.

The coupling device according to the invention can advantageously be used in a surgical instrument that can be disassembled and that can be assembled from a modular instrument system. The coupling device provides the force transmission from a movable handle part of a handle device to a force transmission element connected to a distal tool, wherein the modular instrument system can have different shafts, different handle devices and different tools with different force transmission elements adapted to a limit force in each case of the different tools, which can be advantageously combined to form a surgical instrument as required.

Advantageously, the coupling device according to the invention thus provides, compared to the prior art, a reduction in the play when coupling the force transmission element or its connection element with a movable handle part of the handle device and increases the functional reliability of the tool arranged on the distal side. Furthermore, the coupling device according to the invention not only allows engagement with a plurality of different force transmission elements which are adapted to a limit force predetermined for a corresponding tool, but itself provides a certain adaptation of the force transmission ratio between an actuating force applied by a user to the movable handle part and the force which is transmitted to the tool with the movement of the force transmission element.

Between the release position and the engaging position of the coupling element there may be non-engagement or transition positions in which the connection element is not received without play in the recess of the coupling element. This is the case if a cross-sectional dimension of the recess at the height and in the direction of the longitudinal axis is larger than a longitudinal section area of the connection element, and the width of the notch at the height of the longitudinal axis is even smaller than the width of the connection element. Then there is no play-free engagement of the connection element with the coupling element, although the connection element cannot be released from the coupling element.

If the recess in the coupling element has a circular cross section and the connection element is spherical, the “cross-sectional dimension of the recess” and the “longitudinal dimension of the connection element” are defined as the diameters of the recess and of the connection element, respectively.

Otherwise, i.e., if the recess in the coupling element has a polygonal cross-section, the “cross-sectional dimension at the height and in the direction of the longitudinal axis” refers to the distance of a proximal inner wall of the recess opposite the notch from the notch opening, or from the inner wall portions on the distal side which delimit the notch and provide a stop for the rear grip of the connection element.

The “longitudinal section dimension of the connection element” is the dimension of the connection element in the direction of the longitudinal axis between proximal and distal contact points or surfaces which, in the engaging position, come to rest on the opposite proximal and distal inner wall portions of the coupling element.

In order to further improve the play-free receiving of the connection element in the recess of the coupling element, according to a further embodiment of the coupling device according to the invention it is provided that a cross-sectional shape of the recess in the coupling element perpendicular to the coupling axis corresponds to a shape of a longitudinal section surface of the connection element perpendicular to the coupling axis.

Thus, according to a preferred embodiment of the coupling device according to the invention, the connection element at the proximal end of the force transmission element can be spherical and the recess in the coupling element can be conical or frustoconical with a circular cross section. The opening side of the recess then corresponds to a base area of the conical or frustoconical shape that has the largest diameter of the recess. The cone tip of the conical shape or the top surface of the frustoconical shape has the smallest diameter of the recess. In order to fit into the recess, the diameter of the spherical connection element is between the largest and smallest diameter of the recess. As a variation of the spherical shape of the connecting element, ellipsoids and ovoids should also be included here, for which the conical or frustoconical recess can be formed with a corresponding elliptical or oval cross-sectional shape, so that the longitudinal section dimension of the connection element or cross-sectional dimension of the recess in the longitudinal direction corresponds to the main axis of the elliptical or symmetry axis of the oval cross section.

In principle, however, it is also possible for the cross-sectional shape of the recess in the coupling element perpendicular to the coupling axis to deviate from the shape of the longitudinal section surface of the connection element perpendicular to the coupling axis: For example, a spherical coupling element can also be accommodated in a recess with a polygonal cross-sectional shape. The tapered recess can have a pyramidal or truncated pyramid shape, or a triangular or trapezoidal taper profile in which the proximal and/or distal inner wall portions run towards each other with respect to the coupling axis.

With regard to the course of the tapered recess in the coupling element along the coupling axis, a straight course with respect to the coupling axis may be preferred due to the simpler manufacture, so that, for example in the case of a conical or frustoconical recess, the coupling axis of the coupling element corresponds to the axis of rotation of the recess. However, it is also possible for the tapered recess in the coupling element to run obliquely along the coupling axis, so that, for example, the axis of rotation of the obliquely conical or frustoconical recess does not correspond to the coupling axis of the coupling element. In addition, although a continuous and constant taper of the recess may be preferred, the cross section of the recess can also taper in portions along the coupling axis and/or with different inclinations. The same applies to the notch.

Since the tapered recess of the coupling element has different cross-sectional dimensions and the correspondingly tapered notch has different widths, different force transmission elements, the connection elements of which have different longitudinal dimensions and widths, can be connected to the movable handle part in a simple manner without play by the coupling device according to the invention. All that is required is for the coupling element to be moved along the coupling axis into an engaging position in which the recess has a fitting cross-sectional dimension in the longitudinal direction and the notch has a suitable width for the corresponding connection element.

Accordingly, according to a further embodiment, the coupling device according to the invention can be selectively engaged with a first force transmission element or with a second force transmission element (or with further force transmission elements), each of which has an offset connection element. The first force transmission element, which is configured to transmit a first limit force, has a first connection element, and the second force transmission element, which is configured to transmit a second limit force that is greater than the first limit force, has a second connection element. The longitudinal section dimension and the width of the second connection element are larger than the longitudinal section dimension and the width of the first connection element. In addition, the step width of the second force transmission element can be larger than the step width of the first force transmission element. The same applies to further force transmission elements which are configured to transmit a further limit force with a further connection element, the longitudinal section dimension and width of which are designed depending on the further limit force.

The coupling element can be arranged along the coupling axis for engagement with the first force transmission element in a first engaging position and for engagement with the second force transmission element in a second engaging position which differs from the first engaging position.

In the first engaging position, the cross-sectional dimension of the recess corresponds at the height and in the direction of the longitudinal axis to the longitudinal section dimension of the first connection element and the width of the notch is smaller than the width of the first connection element, so that the first connection element engages behind the notch and comes to rest in the recess. In the second engaging position, the cross-sectional dimension of the recess corresponds at the height and in the direction of the longitudinal axis to the longitudinal section dimension of the second connection element and the width of the notch is smaller than the width of the first connection element, so that the second connection element engages behind the notch and comes to rest in the recess. Since the second connection element is larger than the first connection element, in the engaged position the second connection element rests on a recess cross section that is closer to the opening side of the recess than the recess cross section against which the second connection element rests in the engaged position. Consequently, the coupling element, which is movable along the coupling axis relative to the longitudinal axis, when engaged with the second force transmission element is received further into the slider element in the second engaging position than in the first engaging position.

According to a further embodiment, the coupling device according to the invention can have a bearing component which is in a housing of the handle device, i.e., is arranged therein or connected thereto or, if appropriate, is formed integrally therewith. The bearing component has two guide rails which extend parallel to the longitudinal axis for guiding the slider element in the longitudinal direction. The slider element has, on a side facing away from its guide receptacle, a guide profile with a guide peg formed along the coupling axis, which peg is configured to be received between the two guide rails.

In order to delimit the guide path of the slider element, according to a further embodiment each guide rail can have a guide portion which is limited in the longitudinal direction on both sides by a stop. Accordingly, the slider element can have a support portion on each side adjacent to the guide peg, which portion is mounted in a sliding manner on the guide portion.

Furthermore, according to a further embodiment a coupling device according to the invention can have at least one pivot bracket mounted on the bearing component so as to be pivotable about the pivot axis in order to connect the slider element to the movable handle part. In a preferred embodiment, two parallel oriented pivot brackets pivotably mounted on both sides of the bearing component can be provided for connecting the slider element to the movable handle part for evenly distributed force transmission. For this purpose, a pivot bracket can have a driver portion for the slider element and a connecting portion which is provided for connection to the movable handle part, wherein the pivot axis runs through a central portion between the driver portion and the connecting portion. The, or each, pivot bracket has, in the central portion, a coaxial pivot bearing device to provide the pivot axis, for example a pivot bearing opening or a pivot bearing stub axle. The bearing component has on at least one of the guide rails, preferably on both guide rails, a bearing device coaxial with the pivot axis, which is configured to cooperate with the pivot bearing device for pivotably mounting the pivot bracket. Accordingly, the bearing device can be, for example, an axle stub for cooperation with a pivot bearing opening or a bearing opening for cooperation with a pivot bearing axle stub. Furthermore, both the pivot bearing device and the bearing device can be designed as openings, wherein the cooperation of the pivot bearing opening and the bearing opening is provided by a separate axle element which is received in the openings.

According to yet another embodiment of the coupling device according to the invention, it can be provided that the slider element has a passage opening on the distal side for the passage of the force transmission element. The passage opening is connected to the guide receptacle formed in the slider element and overlaps the notch of the coupling element received in the slider element. The coupling element has at least one outwardly projecting guide pin, preferably two outwardly projecting guide pins, on an axis parallel to the pivot axis. In the slider element there is formed at least one guide gap or preferably two guide gaps, running parallel to the coupling axis and being connected to the guide receptacle. The guide pin or pins are arranged in the guide gap or gaps.

According to yet another embodiment of the coupling device according to the invention, the guide pin or pins are configured to cooperate with the pivot bracket or brackets. For this purpose, the, or each, guide pin protrudes with an end portion from the guide gap of the slider element. The, or each, pivot bracket has a guide groove in the driver portion which runs in the radial direction to the pivot bearing device and in which the end portion of the guide pin is received. Thus, when the handle part is actuated, the pivoting of the pivot bracket is translated into a longitudinal movement of the slider element along the guide rails via the guide pin guided in the guide groove of the pivot bracket and the guide gap of the slider element. Accordingly, the force transmission element connected to the slider element via the coupling element is also moved in the longitudinal direction.

The position of the guide pin axis running parallel to the pivot axis varies with the engaging position of the coupling element and is therefore dependent on the dimensions of the connection element of the force transmission element used, while the position of the pivot axis on the bearing component is fixed. Advantageously, the resulting variable lever ratio allows a certain adjustment of the force ratio of an actuating force applied to the movable handle part to an axial movement force of the force transmission element. The lever ratio consists of a first, constant lever, which is defined between the pivot axis and the handle part on which the actuating force acts, and a second, variable lever, which is defined by the distance of the pivot axis from the guide pin axis when the coupling element is in the engaging position. The second lever is therefore dependent on the dimensions of the connection element which is connected to the coupling element in the engaging position. The variation of the lever ratio is associated with a corresponding variation of the force transmission ratio, which contributes to the protection of other instrument components such as a distal tool. Thus, the lever ratio becomes smaller when smaller connection elements engage with the coupling element, since the coupling element for the engaging position protrudes further from the slider element along the coupling axis and thus the second lever, i.e., the distance between the guide pin axis and the pivot axis, becomes larger. According to the lever ratio, the force for the longitudinal movement of the force transmission element is reduced for the same actuating force on the handle part.

According to a further embodiment of the coupling device according to the invention, this device has a spring element which is supported on a base of the guide receptacle of the slider element and exerts a force on the coupling element in the direction of the coupling axis in order to hold the coupling element in the engaging position. The spring element can be for example a mechanical compression or coil spring, or can be designed as a pneumatic, hydraulic, or magnetic spring element.

In order to facilitate the arrangement of the spring element, according to a still further embodiment of the coupling device according to the invention it is proposed that the slider element has a receiving recess for the spring element. The receiving recess adjoins the guide receptacle, forming an annular step. Furthermore, the receiving recess, which is dimensioned according to the arrangement or receiving of the spring element and is formed in the direction of the coupling axis, then has the base on which the spring element is supported. Alternatively or additionally, the coupling element can have a connecting piece facing away from the opening side of the recess on the upper side of the coupling element, which connecting piece ensures a defined engagement with the spring element. Thus, the connecting piece can be designed coaxially to the coupling axis, forming an annular stop on the underside of the coupling element. The connecting piece is dimensioned such that it can be at least partially received with the spring element in the receiving recess if this is necessary for a corresponding arrangement of the coupling element along the coupling axis in an engaging or release position.

According to a further embodiment of the coupling device according to the invention, it is proposed that the bearing component has a block portion on which the guide rails are arranged, which rails extend parallel to the longitudinal axis and away from the block portion at the proximal side. The block portion can provide the distal stop of the guide portion of each guide rail. A through-opening for the force transmission element extends through the block portion along the longitudinal axis, so that the through-opening in the block portion and the passage opening in the slider element overlap.

According to a first embodiment, a handle device according to the invention for a surgical instrument is provided for arrangement at a proximal end of a shaft through which a force transmission element extends that defines a longitudinal axis and has an offset connection element at a proximal end. The handle device has a handle part that is movable about a pivot axis and a coupling device according to the invention for connecting the movable handle part to the force transmission element.

The surgical instrument capable of being disassembled, equipped with the coupling device according to the invention, can be composed of a modular instrument system in which the coupling device provides the transmission of force from the movable handle part of a handle device according to the invention to a force transmission element connected to a distal tool. As stated, the modular instrument system can have different shafts, different handle devices, and different tools with different force transmission elements adapted to a limit force of each of the different tools, which can be combined as required to form a surgical instrument.

According to a first embodiment, a surgical instrument according to the invention has a shaft through which a force transmission element movable along a longitudinal axis extends, having an offset connection element at a proximal end. The surgical instrument has, at a proximal end of the shaft, a handle device with a handle part that is movable about a pivot axis running perpendicular to the longitudinal axis, and, at a distal end of the shaft, a tool that is operatively connected to the force transmission element. Furthermore, the surgical instrument has a coupling device according to the invention for connecting the movable handle part to the force transmission element.

A method according to the invention for assembling a surgical instrument with a handle device and a force transmission element with an offset connection element at a proximal end is carried out using a coupling device according to the invention. According to a first embodiment, the method according to the invention comprises the following steps:
arranging the coupling element in the guide receptacle of the slider element along the coupling axis in the release position in which, at the height of the longitudinal axis, the width of the notch is greater than the width of the connection element;
inserting the connection element at the proximal end of the force transmission element along the longitudinal axis into the coupling device through the notch in the recess (basically until the connection element passes through the notch in the recess and reaches or crosses the coupling axis),
transferring the coupling element into the engaging position in which, at the height of the longitudinal axis, the cross-sectional dimension of the recess in the direction of the longitudinal axis corresponds to the longitudinal section dimension of the connection element and the width of the notch is smaller than the width of the connection element, so that the connection element engages behind the notch and comes to rest in the recess.

The surgical instrument that can be assembled according to the method can be composed of a modular instrument system.

According to a further embodiment of the method according to the invention for assembling a surgical instrument, it is proposed that the force transmission element configured to transmit a predetermined limit force, is selected according to a maximum limit force predetermined for a tool which is arranged at a distal end of the force transmission element. The assembly of a surgical instrument therefore also comprises connecting the tool to the selected force transmission element, which is adapted to a limit force predetermined for the tool. Further assembly steps include inserting the force transmission element with the proximal-side connection element through a shaft into the handle device, on which the tool is arranged on the distal side and the handle device on the proximal side.

A method also according to the invention for disassembling a surgical instrument with a handle device and a force transmission element with an offset connection element at a proximal end is carried out according to a first embodiment using a coupling device according to the invention, and comprises the following steps:
transferring the coupling element from the engaging position, in which, at the height of the longitudinal axis, the cross-sectional dimension of the recess in the direction of the longitudinal axis corresponds to the longitudinal section dimension of the connection element and the width of the notch is smaller than the width of the connection element, to the release position, in which, at the height of the longitudinal axis, the width of the notch is greater than the width of the connection element; and
removing the connection element from the recess of the coupling element through the notch by pulling the force transmission element out along the longitudinal axis.

Further embodiments, as well as some of the advantages associated with these and other embodiments, are made apparent and better understood from the following detailed description with reference to the accompanying figures. Objects or parts thereof which are substantially the same or similar may be provided with the same reference signs. The figures are merely a schematic representation of an embodiment of the invention. The drawings, the description, and the claims contain numerous features in combination. It is understood that the features mentioned above and those to be explained below can be used not only in the combination specified in each case, but also in other combinations or on their own, without departing from the scope of the present invention.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a partially sectioned side view of a surgical instrument according to the invention with a schematically indicated coupling device according to the invention;

FIG. 2 is a side view of a handle device according to the invention for a surgical instrument with a schematically indicated coupling device according to the invention;

FIG. 3 is a perspective view of a coupling device according to the invention;

FIG. 4 is a plan view of the coupling device from FIG. 3 without force transmission element;

FIG. 5 is an exploded view of the coupling device from FIG. 3;

FIG. 6 is a longitudinal sectional view through the coupling device along section line AA from FIG. 4 with a first force transmission element;

FIG. 7 is a longitudinal sectional view through the coupling device along section line AA from FIG. 4 with a second force transmission element;

FIG. 8 is a longitudinal sectional view according to FIG. 6 with the first force transmission element actuated;

FIG. 9 is a longitudinal sectional view according to FIG. 7 with the second force transmission element actuated;

FIG. 10 is a side view of the coupling device from FIG. 3 in a first engaging position;

FIG. 11 is a side view of the coupling device from FIG. 3 in a second engaging position;

FIG. 12 is a cross-sectional view through the coupling device along section line BB from FIG. 10;

FIG. 13 is a cross-sectional view according to FIG. 12 with force transmission element;

FIG. 14 is a front view of the partially sectioned slider element with coupling element and pivot brackets of the coupling device from FIG. 3;

FIG. 15 is a sectional view in a plane perpendicular to the coupling axis through a connection element located in the recess of a coupling element according to a preferred embodiment in a) engaging position, b) transition position and c) release position; and

FIG. 16 is a sectional view in a plane perpendicular to the coupling axis through a connection element located in the recess of a coupling element according to an alternative embodiment in a) engaging position and b) release position.

### DESCRIPTION OF PREFERRED EMBODIMENTS

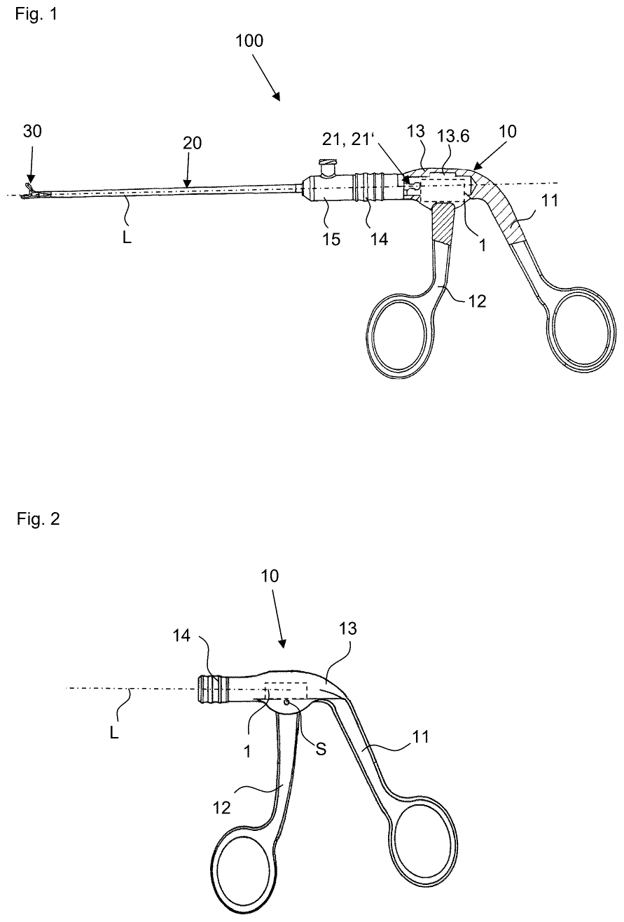

Referring to the drawings, FIG. 1 shows a surgical instrument 100 capable of being disassembled according to the invention, which is composed of a handle device 10 with a coupling device 1 according to the invention (there only shown as a dashed box), a shaft 20 and a tool 30 with a force transmission element 21, 21′. The handle device 10, also shown in FIG. 2, is connected to a proximal end portion 15 of the shaft 20. For this purpose, the handle device 10 shown there has a connecting portion 14 which is formed on a housing 13 of the handle device 10. In this housing 13, there is a receiving space 13.6 for the coupling device 1, which on the one hand is operatively connected to a handle part 12 which is mounted in the housing 13 so as to be pivotable about a pivot axis S and which can be moved relative to a fixed handle part 11 connected to the housing 13.

On the other hand, the coupling device 1 is engaged with the force transmission element 21, 21′ which extends through the shaft 20 along the longitudinal axis L to the tool 30 which is arranged at the distal end of the shaft 20. Thus, an actuation of the handle device 10 on the handle parts 11, 12 is converted by the coupling device 1 into a movement of the force transmission element 21, 21′ along the longitudinal axis L. The coupling device 1 also ensures that the force ratio of the actuating force applied to the handle parts to the force transmitted by the force transmission element can be adapted.

With the longitudinal movement of the force transmission element 21, 21′, the force thereby transmitted causes the actuation of the tool 30. In the example shown in FIGS. 1 and 2, the housing 13 delimiting the receiving space 13.6 with the connecting portion 14 is made in one piece with the fixed handle part 11. Not shown are alternative embodiments of handle devices 10 according to the invention, which are composed of two or more housing components which, when assembled, can correspond to the illustrated one-piece housing component 13. Other handle devices may differ from the example shown with regard to the connecting portion at which the handle device is connected to the shaft. This in turn can be formed without such a proximal end portion 15, or by an alternatively designed proximal end portion for connection to a handle device.

The tool 30 shown in FIG. 1 is a gripping tool with two jaw parts, which has a mechanism not explained in detail here via which the tool 30 is connected to the force transmission element 21, 21′ and which converts its longitudinal movement into tool movements. In this case, these tool movements are opening and closing movements of the jaw parts. Of course, other tools with deviating functions which are connected to a force transmission element can also be used in a surgical instrument according to the invention.

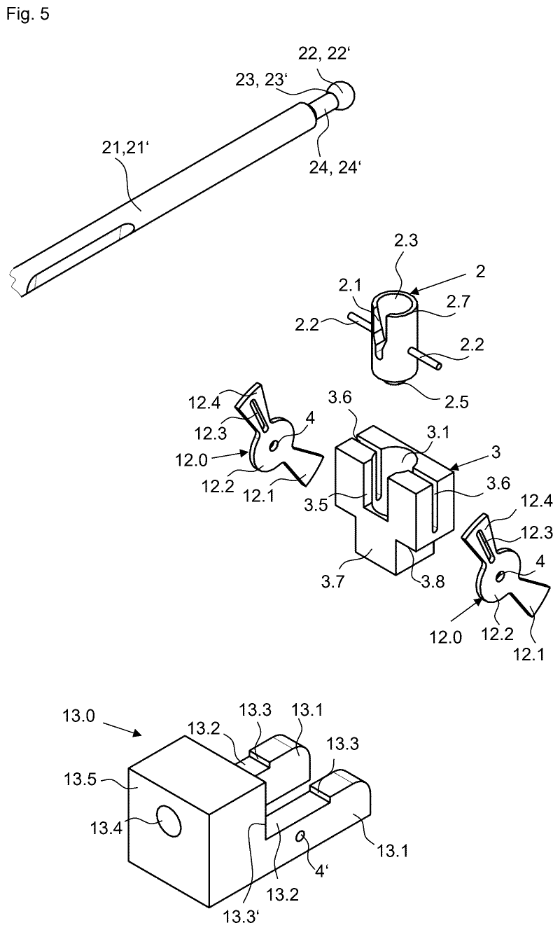

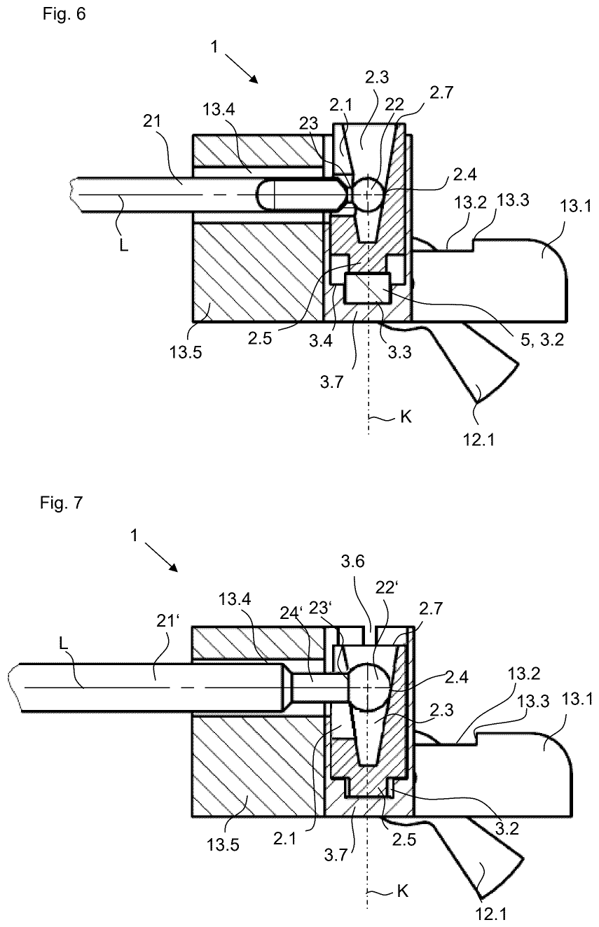

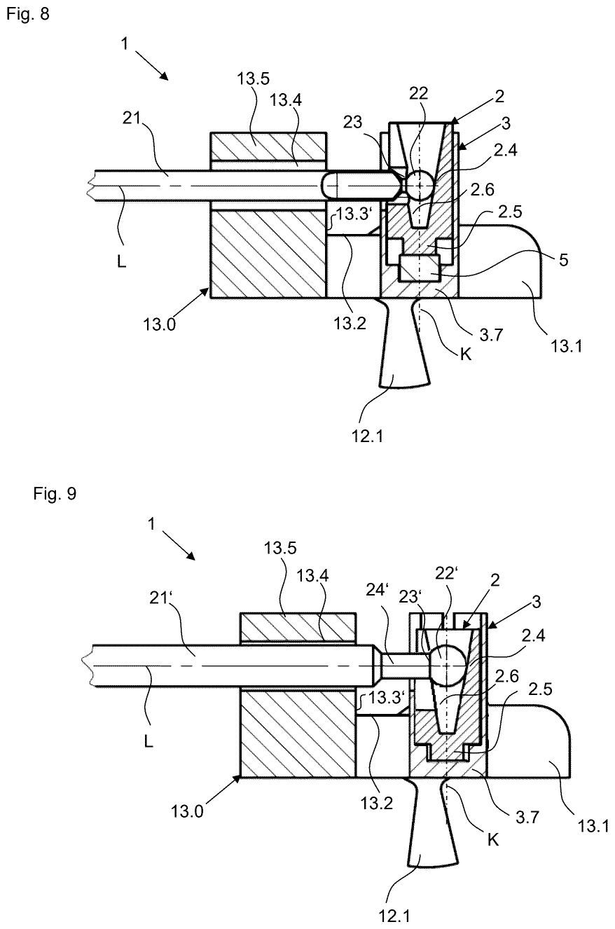

The force transmission element 21, 21′, which can in each case engage with the coupling device 1 and of which a portion with the connection element 22, 22′ at the proximal end can be seen in FIG. 5, can optionally be a first force transmission element 21 (FIGS. 6, 8) or a second force transmission element 21′ (FIGS. 7, 9) (or a further force transmission element not shown), which differ with regard to the force they can transmit. The present force transmission elements 21, 21′ are designed as pull and push rods, wherein the force transmission elements 21, 21′ can be designed over their length substantially with a uniform, for example circular, cross-sectional profile with a constant diameter. However, a force transmission element 21, 21′ can also have, for example, flattened portions, as shown in FIG. 5, or portions with a differently deviating cross section, if these are advantageous or desirable for the cooperation with an instrument component located further on the distal side.

The first, second, and optionally each further force transmission element 21, 21′ differs at least with regard to its thickness, or its diameter, as well as a cross-sectional dimension of each of the connection elements 22, 22′, as can be seen for example by comparing FIGS. 6 and 7, which show the same coupling device 1 in engagement with a first force transmission element 21 and a second force transmission element 21′. Furthermore, the force that can be transmitted by the force transmission element 21, 21′ can be influenced for example by the choice of material.

Which force transmission element 21, 21′ is used in a surgical instrument 100 from FIG. 1 depends on the tool 30 used: This is because the tools available in a modular instrument system differ in terms of their load limit, depending on the material and intended use. The maximum tolerable limit force predetermined for each tool should not be exceeded in order to maintain the functionality of the tool and to avoid damage or breakage of the tool. For example, the predetermined limit force for tools for cutting or punching bone tissue is significantly higher than the predetermined limit force for tools for preparing softer tissue. Accordingly, a force transmission element that is intended for connection to a tool for cutting or punching bone tissue will be configured to transmit a higher force and will therefore have a greater thickness, or a larger diameter (like the second force transmission element 21′), than a force transmission element that is intended for connection to a tool for preparing softer tissue and is therefore configured to transmit a lower force and accordingly has a smaller thickness or a smaller diameter (than the first force transmission element 21).

Therefore, the tool 30 and force transmission element 21, 21′ may, but need not, be provided as suitably assembled units in a modular instrument system. A modular instrument system can also have separate tools and force transmission elements that can be assembled in a suitable manner.

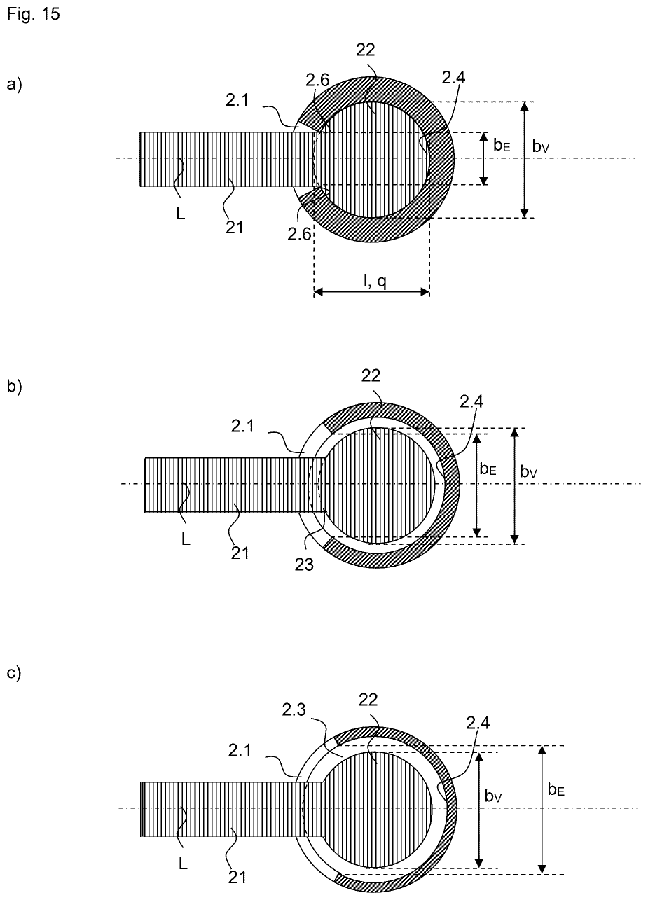

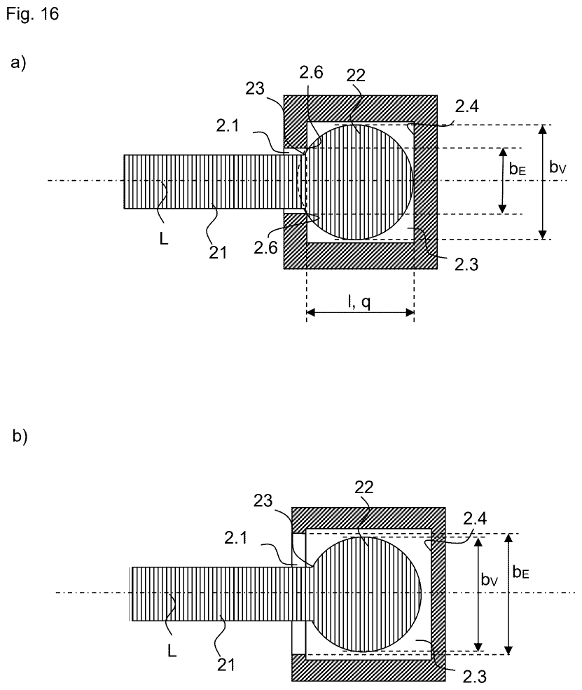

For engagement with the coupling device 1 according to the invention, each force transmission element 21, 21′ has a connection element 22, 22′ at its proximal end, which is delimited from the rod-shaped force transmission element 21, 21′ by a step 23, 23′. In preferred embodiments, the connection element 22, 22′ is spherical, as shown in FIGS. 5 to 9. The step 23, 23′ to the spherical connection element 22, 22′ is provided here by a neck portion 24, 24′ of the force transmission element 21, 21′ with reduced cross-sectional dimensions. The neck portion 24, 24′ has a diameter that is smaller than the diameter of the connection element 22, 22′ and of the remaining rod-shaped force transmission element 21, 21′. In the embodiments shown, the force transmission element 21, 21′ has the same diameter as the connection element 22, 22′, apart from the neck portion 23, 23′. However, differing from this, a step 23 can also be formed without a neck portion if the diameter of the connection element 22 is larger than the diameter of the force transmission element 21 (see FIGS. 15 and 16).

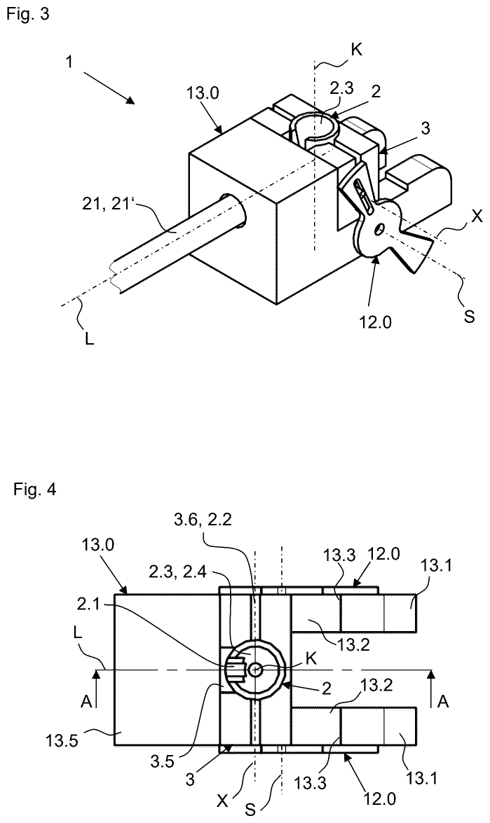

The coupling device 1 according to the invention, as shown in FIGS. 3 to 14, is provided for coupling such a force transmission element 21, 21′, which defines a longitudinal axis L, to a handle part 12 of the surgical instrument 100 which is movable about a pivot axis S perpendicular to the longitudinal axis L. For this purpose, the coupling device 1 has a coupling element 2 and a slider element 3 cooperating therewith. The coupling element 2 has a recess 2.3 for receiving the offset connection element 22, 22′, and the slider element 3 is configured to cooperate with the movable handle part 12 and to guide it in the direction of the longitudinal axis L.

The slider element 3 has a guide receptacle 3.1 for receiving the coupling element 2, which receptacle is formed along a coupling axis K which runs perpendicular to the longitudinal axis L and perpendicular to the pivot axis S. Thus, the coupling element 2 can be moved along the coupling axis K in the upwardly open guide receptacle 3.1 of the slider element 3. To guide this movement, the inner contour of the guide receptacle 3.1 corresponds at least partially with the outer contour of the coupling element 2. In the coupling element 2, the recess 2.3 for receiving the connection element 22, 22′, which is at the proximal end of the force transmission element 21, 21′, also runs along the coupling axis K.

The cross section of the recess 2.3 tapers starting from the upwardly facing opening side 2.7 along the coupling axis K. In addition, the coupling element 2 has a notch 2.1 on the distal side which is connected to the recess 2.3 and also tapers in a direction parallel to the coupling axis K. The notch 2.1 thus has its greatest width be on the opening side 2.7 (see FIG. 14). The width be of the notch 2.1 parallel to the coupling axis K can for example decrease constantly, or, as can be seen in FIG. 14, only in portions in a central region of the notch. Alternatively, the tapering of the notch can also be non-constant and/or made in multiple portions.

Since the coupling element 2 is received in the guide receptacle 3.1 of the slider element 3, the guide receptacle 3.1 of the slider element 3 is connected on the distal side to a passage opening 3.5 which overlaps the notch 2.1 of the coupling element 2 for the passage of a force transmission element 21, 21′ with connection element 22, 22′.

The dimensions of the tapered cross section of the recess 2.3 and of the tapered notch 2.1 of the coupling element 2 are dimensioned such that differently sized connection elements 22, 22′ of different force transmission elements 21, 21′ can be received in the recess 2.3. For this purpose, the coupling element 2 in the coupling device 1 is movable perpendicular to the longitudinal axis L of the force transmission element 21, 21′ along the coupling axis K in the guide receptacle 3.1 of the slider element 3. In this way, the cross-sectional dimensions of the recess 2.3 and the width of the notch 2.1 at the height of the longitudinal axis L vary depending on the position of the coupling element 2 along the coupling axis K in the guide receptacle 3.1.

For a given force transmission element 21, 21′ with a connection element 22, 22′ having defined dimensions, the coupling element 2 can be moved along the coupling axis K in the guide receptacle 3.1 between an engaging position in which the connection between the force transmission element 21, 21′ and the handle part is established and a release position in which the force transmission element 21, 21′ is not connected to the handle part.

In the engaging position of the coupling element 2, as can be seen in particular in FIGS. 15a and 16a, at the height of the longitudinal axis L the cross-sectional dimension q of the recess 2.3 in the direction of the longitudinal axis L corresponds to the longitudinal section dimension 1 of the connection element 22, which thus comes to rest against the inner wall 2.4 opposite the notch 2.1. Furthermore, at the height of the longitudinal axis L, the width be of the notch 2.1 is smaller than the width b_{V} of the connection element 22, so that the connection element 22 engages behind the notch 2.1 and comes to rest in the recess 2.3 on the inner wall portions 2.6 which delimit the notch 2.1. In this way, the connection element 22 engages with the coupling element 2 without play in the longitudinal direction. The same applies to the coupling devices 1 shown in FIGS. 6 to 9, 12, which engage with different connection elements 22, 22′ of different force transmission elements 21, 21′.

Since FIG. 15 shows a spherical connection element 22 and a recess 2.3 with a circular cross-section, by definition the longitudinal section dimension 1 of the connection element 22 and the cross-sectional dimension q of the recess 2.3 at the height and in the direction of the longitudinal axis L in the engaging position in FIG. 15a correspond to the diameter of the recess 2.3 and of the connection element 22 at the height of the longitudinal axis L.

FIG. 16, on the other hand, shows a spherical connection element 22 which is received in a recess 2.3 with a polygonal, here rectangular, cross-section. The recess 2.3 with such a polygonal cross-sectional shape can for example be shaped as a truncated pyramid in which all side walls converge towards each other. Alternatively, it is also possible that only the proximal inner wall 2.4 opposite the notch and the inner wall portions 2.6 delimiting the notch converge towards each other, so that the recess has a trapezoidal taper profile. There, the cross-sectional dimension q of the recess 2.3 corresponds to the distance in the longitudinal direction L between the proximal inner wall 2.4, which is opposite the notch 2.1, and the distal inner wall portions 2.6, which delimit the notch 2.1. This distance is dimensioned at the height of the longitudinal axis L for a given spherical connection element 22 such that the spherical connection element 22 comes to rest on the proximal inner wall 2.4 on the proximal side and on the distal inner wall portions 2.6 on the distal side. In the example shown, the connection element 22 contacts the distal inner wall portions 2.6 at the proximal side of the step 23 of the point of the connection element 22 whose width corresponds to the width be of the notch 2.1, which is greater than the width of the step 23.

FIGS. 15c and 16b each show a corresponding release position of the coupling element 2, in which at the height of the longitudinal axis L the width be of the notch 2.1 is greater than the width b_{V} of the connection element 22, so that the connection element 22 can be pulled out through the notch 2.1 in the direction of the longitudinal axis L. Furthermore, by comparison with FIGS. 15a and 16a, it can be seen that the cross-sectional dimensions of the recess 2.3 are larger in the release position of the coupling element 2 than in the engaging position. To transfer from the engaging position in FIGS. 15a and 16a to the release position according to FIGS. 15c and 16b, the coupling element 2 is moved perpendicular to the plane of the drawing.

In FIG. 15b, a non-engagement or transition position of the coupling element 2 is also shown, which lies between the engagement and release positions from FIG. 15a, c, in which the cross-sectional dimensions of the recess 2.3 at the height of the longitudinal axis L are larger than the longitudinal section dimensions of the connection element 22. However, since the width be of the notch 2.1 at the height of the longitudinal axis L is smaller than the width b_{V} of the connection element 22, the connection element 22 cannot yet be pulled out through the notch 2.1, although the connection element 22 no longer rests in the recess 2.3 without play in the longitudinal direction. In a modification of the coupling device 1, in which the coupling element in the release position is moved along the coupling axis K so far away from the longitudinal axis L that the connection element is above the opening side, i.e., no longer in the recess, the notch width on the opening side of the coupling element can be smaller than the width of the connection element.

Particularly advantageous are the embodiments of the coupling device 1 shown in FIGS. 3 to 15, in which the recess 2.3 in the coupling element 2 is frustoconical with a circular cross section for engagement with a spherical connection element 22, 22′. The cross-sectional dimension q of the recess 2.3 in the direction of the longitudinal axis L then corresponds to the diameter of the circular cross-section. Also, the longitudinal section dimension 1 of the connection element 22, 22′ corresponds to the diameter of the spherical connection element 22, 22′. This allows the connection element 22, 22′ to be accommodated in the recess 2.3 of the coupling element 2 without play, without risk of the connection element 22, 22′ jamming, since the connection element 22, 22′ does not come to rest in the recess with a flat surface, but only along a circumferential line.

In the embodiment shown in FIGS. 3 to 14, a bearing component 13.0 of the coupling device 1 is designed with two guide rails 13.1 which extend parallel to the longitudinal axis L in order to guide the slider element 3 in the longitudinal direction. The two guide rails 13.1 extend from a block portion 13.5 of the bearing component 13.0, which has a through-opening 13.4 along the longitudinal axis L, through which the force transmission element 22, 22′ extends in longitudinally movable fashion. The bearing component 13.0 of the coupling device 1 is correspondingly provided to be arranged in the housing 13 of the handle device 10 of the surgical instrument 100.

The slider element 3 has a guide peg 3.7 on a side facing away from the guide receptacle 3.1, which is designed as a guide profile along the coupling axis K between two support portions 3.8. The width of the cuboid-shaped guide peg 3.7 corresponds to the distance between the guide rails 13.1, so that the guide peg 3.7 can be received between the guide rails 13.1. The support portions 3.8 offset on both sides of the peg 3.7 are configured to correspond to the guide rails 13.1 and lie in a sliding manner on the guide rails 3.1. Each guide rail 13.1 has a guide portion 13.2 which defines a sliding path for the slider element 3, the length of which is limited by a proximal stop 13.3 and a distal stop 13.3′. In the example shown, the distal stop 13.3′ is formed by the block portion 13.5.

For connection to the movable handle part 12, the coupling device 1 in the embodiments shown in FIGS. 3 to 14 has two pivot brackets 12.0 mounted on both sides of the bearing component 13.0 so as to be pivotable about the pivot axis S. Each pivot bracket 12.0 has a connecting portion 12.1 for connection to the movable handle part 12 and a driver portion 12.4 to transmit the movement of the handle part 12 to the slider element 3. Between the connecting portion 12.1 and the driver portion 12.4 there is a central portion 12.2 which, as a pivot bearing device, has a pivot bearing opening 4 through which the pivot axis S runs. The connecting portion 12.1 and the driver portion 12.4 run in the radial direction to the pivot bearing opening 4 and, in the example shown, are arranged at an angle of approximately 160° to each other. Differing from this, however, a diametrical arrangement of the connecting portion and the driver portion on the central portion or an angled arrangement with an angle deviating from 160° are also possible; this depends, among other things, on the length of the sliding path and the design and arrangement of the handle part 12.

In a region below the guide portion 13.2, each guide rail 13.1 has a bearing opening 4′ as a bearing device, as can be seen in FIG. 5, which is designed coaxially to the pivot axis S for cooperation with the pivot bearing device 4 so that the pivot bracket 12.0 can be pivotably connected to the bearing component 13.0 about the pivot axis S. Not shown in the figures is an axle element that cooperates with the pivot bearing opening 4 and the bearing opening 4′ to form the pivot joint. For this purpose, an axle element can, for example, be arranged with one axle end either in the pivot bearing opening 4 or the bearing opening 4′ in a rotationally fixed manner and with the other axle end rotatably mounted in the other opening, i.e., the bearing opening 4′ or the pivot bearing opening 4. Alternatively, the axle element can be designed as a free-running axle and be rotatably mounted in both openings, the bearing opening 4′ and the pivot bearing opening 4. Deviating from the example shown, in a variant one of the bearing and pivot bearing devices can be designed as an axle stub instead of an opening, which is rotatably mounted in the other bearing or pivot bearing device designed as an opening.

The cooperation of the pivot bracket 12.0 with the slider element 3 is provided via the coupling element 2 received in the slider element 3. For this purpose, the coupling element 2 has two diametrically outwardly projecting guide pins 2.2 which define an axis X parallel to the pivot axis S (see FIGS. 3, 4, 10). For these two guide pins 2.2, the slider element 3 has two guide gaps 3.6 which are diametrically connected to the guide receptacle 3.1 and lie in a plane defined by the axis X and the coupling axis K. Thus, both guide pins 2.2 are guided in the guide gaps 3.6 in a direction parallel to the coupling axis K when the coupling element 2 is moved in the guide receptacle 3.1 along the coupling axis K.

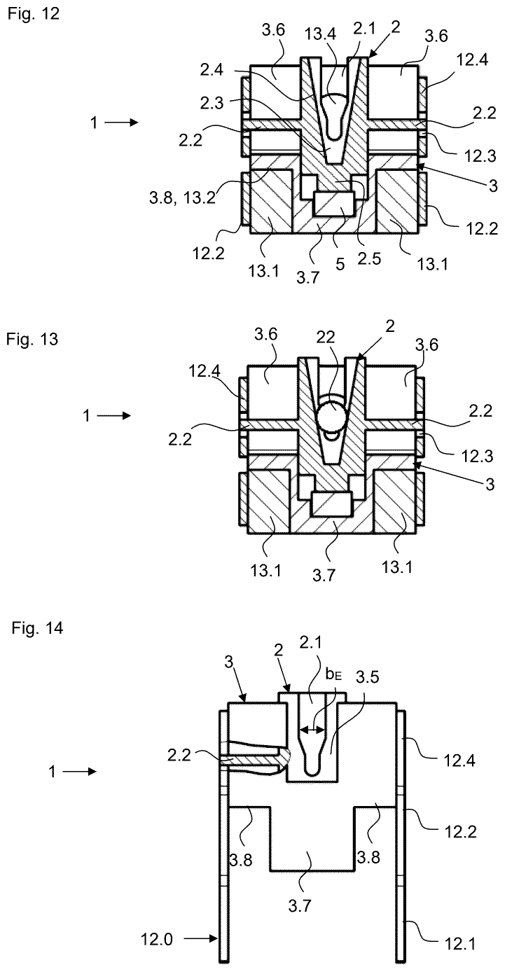

The guide pins 2.2 are dimensioned such that they protrude with an end portion at their free end from the guide gap 3.6 of the slider element 3, as can be seen in FIG. 3 and in particular in FIGS. 12 to 14. The free end portion of the guide pins 2.2 protrudes into a guide groove 12.3 which is formed in the pivot bracket 12.0. This guide groove 12.3 extends in the driver portion 12.4 in the radial direction to the pivot bearing device 4, as can be seen in FIGS. 3, 5 and 10. When the handle part 12 is actuated, the pivot bracket 12.0 is moved about the pivot axis S, wherein the pivot movement of the driver portion 12.4 is converted into a longitudinal movement of the slider element 3 via the coupling element 2 received therein via the guide pin 2.2 received in the guide groove 12.3.

This is shown in FIGS. 6 to 9 for the coupling device 1 engaged with two different force transmission elements 22, 22′: FIGS. 6 and 7 show the slider element 3 at the distal stop 13.3′ of the guide portion 13.2 of the guide rail 13.1, wherein the connecting portion 12.1 of the pivot bracket 12.0 is pivoted in the proximal direction. After actuation of the movable handle part 12, the connecting portion 12.1, connected thereto, in FIGS. 8 and 9 is pivoted in the distal direction. In this case, the driver portion 12.4, which is covered by the slider element 3 and the coupling element 2 in FIGS. 6 to 9, is pivoted accordingly in the proximal direction. This movement of the driver portion 12.4 leads to the longitudinal movement of the slider element 3 along the guide rails 13.1 via the coupling of the coupling element 2 with both the driver portion 12.4 and the slider element 3.

The force transmission element 21, 21′, engaging in each case with the coupling element 2, follows the movement in the longitudinal direction, whereby a tool 30 at the distal end of a surgical instrument 100 (cf. FIG. 1) is actuated, for example for closing the jaws of preparing, grasping, and excision forceps, punches and scissors. Depending on the size and area of application, different limit forces are specified for these tools, which should not be exceeded in order to avoid component failure or breakage. Therefore, surgical instrument systems contain different force transmission elements that are configured to couple with the tools to transmit different limit forces in order to prevent overloading of the distal tool.

For this purpose, two different force transmission elements 21, 21′ are shown in FIGS. 6 to 9, which are configured to transmit different limit forces and are in engagement with the coupling element 2 of the coupling device 1. The first force transmission element 21 shown in FIGS. 6 and 8 is configured to transmit a first limit force which is smaller than the second limit force that can be transmitted with the second force transmission element 21′ seen in FIGS. 7 and 9. Both force transmission elements 21, 21′ have a spherical connection element 22, 22′ at their proximal end, which is offset from the rod-shaped force transmission element 21, 21′ by a neck portion 23, 23′ with a reduced diameter. The first force transmission element 21, which is configured to transmit the smaller limit force, has a smaller diameter than the second force transmission element 21′, wherein the first spherical connection element 22 correspondingly has a smaller diameter than the second spherical connection element 22. Also, since the engaging position of the coupling element 2 along the coupling axis K depends on the fact that the cross-sectional diameter of the recess 2.3 at the height of the longitudinal axis L corresponds to the diameter of the connection element 22, 22′, the engaging position of the coupling element 2 with the first connection element 22 along the coupling axis K also differs from the engaging position of the coupling element 2 with the second connection element 22.

FIGS. 6 and 8 show the coupling element 2 in a first engaging position with the first, smaller connection element 22, while in FIGS. 7 and 9 the coupling element 2 is shown in a second engaging position with the second, larger connection element 22′. Since the recess 2.3 tapers in the shape of a frustum, the coupling element 2 is moved further in the direction of the longitudinal axis L along the coupling axis K for the first engaging position than for the second engaging position. In the first engaging position, in which it comes to rest on the proximal inner wall portion 2.4 and engages behind the notch 2.1 on the distal inner wall sections 2.6, the first, smaller connecting element 22 is thus further away from the opening side of the recess 2.3 than the second, larger connection element 22. This second connection element is in the second engaging position, in which it comes to rest on the proximal inner wall portion 2.4 and engages behind the notch 2.1 on the distal inner wall portions 2.6, closer to the opening side of the recess 2.3.

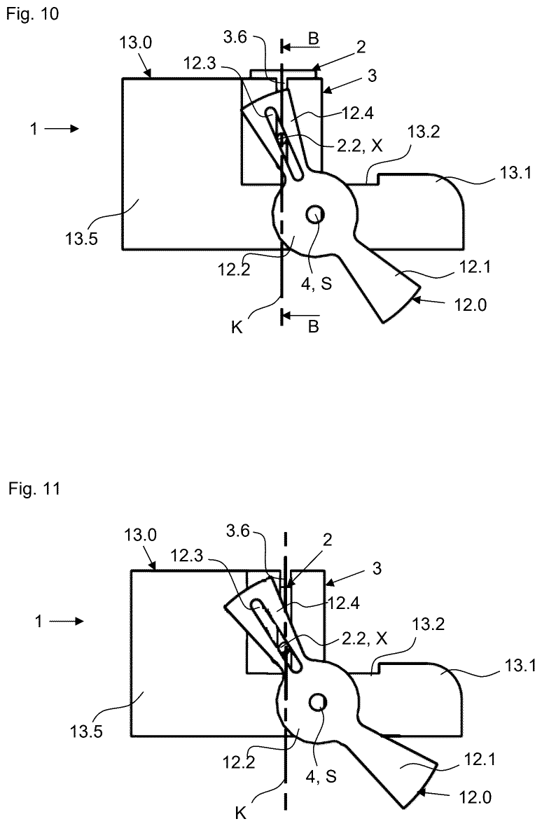

With respect to the slider element 3, the coupling element 2 is received further in the guide receptacle 3.1 in the second engaging position than in the first engaging position. This means that the distance between the pivot axis S and the axis X, which is defined by the guide pins 2.2 of the coupling element 2, is greater in the first engaging position (FIG. 10) than in the second engaging position (FIG. 11). Thus, the position of the guide pin axis X in relation to the parallel pivot axis S varies depending on the dimensions of the connection element 22, 22′ of the force transmission element 21, 21′ used. However, the position of the pivot axis S on the bearing component 13.0 and the dimensions of the connecting portion 12.1 of the pivot bracket 12.0 and the associated handle part 12 are fixed.

With respect to the pivot axis S, there is a lever ratio between a first, constant lever, which is defined between the pivot axis S and the handle part 12 on which the actuating force acts, and a second, variable lever, which is defined by the distance of the pivot axis S from the guide pin axis X when the coupling element 2 is in the engaging position. This lever ratio changes with the engaging position, which varies depending on the dimensions of the connection element 22, 22′, whereby the force ratio of the actuating force applied to the movable handle part to an axial movement force of the force transmission element 21, 21′ can be adjusted to a certain extent.

When the smaller connection element 22 engages with the coupling element 2 in the first engaging position (FIG. 10), the second lever is larger than when the larger connection element 22′ engages with the coupling element 2 in the second engaging position (FIG. 11). Consequently, the lever ratio between the constant first lever and the second lever is smaller in the first engaging position (FIG. 10) than in the second engaging position (FIG. 11). Accordingly, with the same actuating force on the handle part 12, a smaller force for longitudinal movement is transmitted to the smaller force transmission element 21 in the first engaging position than to the larger force transmission element 21′ in the second engaging position. This provides additional protection for distal components such as the tool and ensures its functionality.

Also, in order to hold the coupling element 2 in the engaging position, the coupling device 1 has a spring element 5, seen for example in FIGS. 6, 8, and 12.

The spring element 5 exerts a force on the coupling element 2 directed in the direction of the coupling axis K, which force presses the coupling element 2 upwards in the guide receptacle 3.1. The spring element 5 in the slider element 3 is supported at the base 3.3 in a receiving recess 3.2, which adjoins the guide receptacle 3.1 so as to form an annular step 3.4. The coupling element 2 has a connecting piece 2.5 to which a spring element 5 such as a coil spring can be secured. The connecting piece 2.5 is offset on the underside of the coupling element 2 coaxially to the coupling axis K so as to form a shoulder, and is configured to correspond to the receiving recess 3.2. Depending on the position of the engaging position, the connecting piece 2.5 can thus be partially or entirely received in the receiving recess 3.2, with the spring element 5 being compressed.

Therefore, the coupling device 1 shown in FIGS. 7 and 9 can also have a spring element 5, which, however, cannot be seen in the illustration due to the arrangement of the coupling element 2 in the second engaging position. This is because here the connecting piece 2.5 is almost completely received in the receiving recess 3.2, so that the spring element 5 is maximally compressed.

It is understood that the coupling device 1 according to the invention is also suitable for engagement with other force transmission elements (not shown) with connection elements whose dimensions differ from the dimensions of the connection elements shown, e.g. lie between them. The coupling device 1 can engage with any connection element whose diameter corresponds to a cross-sectional dimension at a point of the recess 2.3 and is larger than the width of the notch 2.1 at that point. When this point of the coupling element 2 comes to lie at the height of the longitudinal axis L, the coupling element 2 has reached an engaging position with the corresponding connection element. This makes it possible to realize a multiplicity of component combinations with regard to the tool and the force transmission element of a surgical instrument, which have a play-free connection of the force transmission element with the handle part. In addition, the lever ratio, which varies with the engaging position, helps to protect the force transmission element and the associated distal tool as well as, possibly, other components from overload when the handle part is actuated with excessive force. Furthermore, assembly and disassembly of a surgical instrument are simplified by a coupling device according to the invention.

To assemble a surgical instrument 100 as in FIG. 1, first a tool 30 and a force transmission element 21, 21′ fitting thereto with an offset connection element 22, 22′ at the proximal end as well as an instrument shaft 20 and a handle device 10 are selected, which handle device has a movable handle part 12 and a coupling device 1, which can correspond to the embodiment from FIGS. 3 to 14. The tool 30 is connected to the distal end of the force transmission element 21, 21′, which is configured to transmit a maximum limit force predetermined for a tool 30. The force transmission element 21, 21′ is inserted through the instrument shaft 20 into the handle device 10 until the connection element 22, 22′ at the proximal end of the force transmission element 21, 21′ reaches the coupling device 1. Before or after this, the instrument shaft 20 can be connected to a tool holder on the distal side and to the handle device 10 on the proximal side.

The coupling element 2 is arranged in the guide receptacle 3.1 of the slider element 3 along the coupling axis K in a release position or is moved into a release position in which, at the height of the longitudinal axis L, the width of the notch 2.1 is greater than the width of the connection element 22, 22′, wherein, if necessary, the diameter, or the cross-sectional dimension, of the recess 3.2 in the direction of the longitudinal axis L is also greater than the diameter, or the longitudinal section dimension, of the connection element 22, 22′. The connection element 22, 22′ at the proximal end of the force transmission element 21, 21′ is then inserted along the longitudinal axis L through the notch 2.1 into the recess 2.3 until the connection element 22, 22′ crosses the coupling axis K. The coupling element 2 can then assume the corresponding engaging position in which, at the height of the longitudinal axis L, the diameter or the cross-sectional dimension of the recess 2.3 in the direction of the longitudinal axis L corresponds to the diameter or the longitudinal section dimension of the connection element 22, 22′ and the width of the notch 2.1 is smaller than the width of the connection element 22, 22′. The connection element 22, 22′ then engages behind the notch 2.1 and comes to rest in the recess 2.3 in the engaging position at least on a proximal inner wall 2.4 opposite the notch 2.1 and on the distal inner wall portions 2.6 which delimit the notch 2.1. The surgical instrument 100 is then ready for use.

The disassembly of the surgical instrument 100 takes place in the reverse order. Here the coupling element 2 is transferred from the engaging position into a release position in which the connection element 22, 22′ can be pulled out of the recess 2.3 of the coupling element 2 and thus out of the handle device 10 along the longitudinal axis L through the notch 2.1. Further disassembly steps can include separating the instrument shaft 20 from the handle device 10 and/or from a distal tool holder and removing the force transmission element 21, 21′ from the instrument shaft 20. If necessary, the tool 30 can also be separated from the distal end of the force transmission element 21, 21′ in order to dispose of the components of the surgical instrument 100 separately or to prepare them for reuse by cleaning and disinfection.

The drawings, the description, and the claims contain numerous features in combination. It goes without saying that the above-mentioned features can be used not only in the combination specified in each case, but also in other combinations or on their own, without departing from the scope of the present invention. The invention relates to a coupling device 1 for a surgical instrument 100, and a handle device 10 for same, as well as corresponding assembly and disassembly methods. The instrument 100 has a force transmission element 21, 21′ defining a longitudinal axis L and having an offset connection element 22, 22′ at a proximal end, and a handle device 10 having a handle part 12 that can move about a pivot axis S running perpendicular to the longitudinal axis L. For connecting the force transmission element 21, 21′ to the handle part 12, the coupling device 1 has a coupling element 2 with a recess 2.3 for receiving the connection element 22, 22′ and a slider element 3 which cooperates with the coupling element 2 and is configured to cooperate with the movable handle part 12 and to move in the direction of the longitudinal axis L. The coupling element 2 is received in a guide receptacle 3.1 of the slider element 3 such that it can move along a coupling axis K, and the recess 2.3 in the coupling element 2 corresponds to the coupling axis K. The coupling element 2 has a notch 2.1 which is connected to the recess 2.3. The coupling element 2 is movable along the coupling axis K between at least one release position and one engaging position.

While specific embodiments of the invention have been shown and described in detail to illustrate the application of the principles of the invention, it will be understood that the invention may be embodied otherwise without departing from such principles.

### LIST OF REFERENCE SIGNS

1 Coupling device
2 Coupling element
2.1 Notch
2.2 Guide pin
2.3 Recess
2.4 Proximal inner wall portion
2.5 Connecting piece
2.6 Distal inner wall portion
2.7 Opening side
3 Slider element
3.1 Guide receptacle for coupling element
3.2 Receiving recess for spring element
3.3 Base
3.4 Step
3.5 Passage opening for force transmission element
3.6 Guide gap for guide pin
3.7 Guide peg
3.8 Support portion
4,4′ Pivot bearing device, bearing device
5 Spring element
10 Handle device
11 Fixed handle part
12 Movable handle part
12.0 Pivot bracket
12.1 Connecting portion
12.2 Central portion
12.3 Guide groove for guide pin
12.4 Driver portion
13 Housing
13.0 Bearing component
13.1 Guide rail
13.2 Guide portion
13.3, 13.3′ Stop
13.4 Through-opening
13.5 Block portion
13.6 Receiving space
14 Connecting portion
15 Proximal shaft end portion
20 Shaft
21, 21′ Force transmission element
22, 22′ Connection element
23, 23′ Step
24, 24′ Neck portion
30 Tool
100 Surgical instrument
L Longitudinal axis
K Coupling axis
S Pivot axis
X Axis guide pins
1, q Longitudinal section dimension, cross-sectional dimension
b_{E}, b_{V} Width of the notch, width of the connection element

1. A coupling device for a surgical instrument, which comprises a force transmission element that defines a longitudinal axis and that comprises an offset connection element at a proximal end; and a handle device, the handling device comprising a handle part that can move about a pivot axis running perpendicular to the longitudinal axis the coupling device comprising:
a coupling element for connecting the force transmission element to the movable handle part, the coupling element comprising a recess for receiving the connection element; and
a slider element which cooperates with the coupling element and is configured to cooperate with the movable handle part and to move in a direction of the longitudinal axis, wherein the coupling element is received in a guide receptacle of the slider element so as to be movable along a coupling axis which runs perpendicular to the longitudinal axis wherein the recess in the coupling element is configured corresponding to the coupling axis and has a cross section which tapers in a direction of the coupling axis, and the coupling element comprises a notch which is connected to the recess, wherein a width of the notch tapers in a direction parallel to the coupling axis, and wherein the coupling element is configured to move along the coupling axis between at least one release position in which at a height of the longitudinal axis the width of the notch is greater than a width of the connection element, and at least one engaging position in which, at the height of the longitudinal axis, a cross-sectional dimension of the recess in the direction of the longitudinal axis corresponds to a longitudinal section dimension of the connection element and the width of the notch is smaller than the width of the connection element, so that the connection element engages behind the notch and comes to rest in the recess.

2. The coupling device according to claim 1, wherein a cross-sectional shape of the recess in the coupling element perpendicular to the coupling axis corresponds to a shape of a longitudinal section surface of the connection element perpendicular to the coupling axis.

3. The coupling device according to claim 1, wherein the coupling device is configured to be brought into engagement with at least one first force transmission element or with a second force transmission element, wherein the first force transmission element comprises an offset first connection element for transmitting a first limit force and the second force transmission element comprises an offset second connection element for transmitting a second limit force which is greater than the first limit force, wherein the longitudinal section dimension of the second connection element is greater than the longitudinal section dimension of the first connection element and the width of the second connection element is greater than the width of the first connection element, and wherein the coupling element is
arranged for engagement with the first force transmission element along the coupling axis in a first engaging position in which, at the height of the longitudinal axis the cross-sectional dimension of the recess in the direction of the longitudinal axis corresponds to the longitudinal section dimension of the first connection element and the width of the notch is smaller than the width of the first connection element so that the first connection element engages behind the notch and comes to rest in the recess, and
arranged with the second force transmission element along the coupling axis in a second engaging position, in which at the height of the longitudinal axis the cross-sectional dimension of the recess in the direction of the longitudinal axis corresponds to the longitudinal section dimension of the second connection element and the width of the notch is smaller than the width of the first connection element, so that the second connection element engages behind the notch and comes to rest in the recess.

4. The coupling device according to claim 1, wherein the coupling device comprises a bearing component which is present in a housing of the handle device, wherein the bearing component comprises two guide rails which extend parallel to the longitudinal axis, and wherein the slider element comprises, on a side facing away from the guide receptacle, a guide profile with a guide peg formed along the coupling axis, which peg is received between the two guide rails.

5. The coupling device according to claim 4, wherein the coupling device comprises at least one, pivot bracket mounted on the bearing component so as to be pivotable about the pivot axis for connection to the movable handle part, wherein the pivot bracket comprises a pivot bearing device for providing the pivot axis, and the bearing component comprises at least one bearing device coaxial with the pivot axis, which is configured to cooperate with the pivot bearing device for pivotably mounting the at least one pivot bracket on at least one of the guide rails.

6. The coupling device according to claim 1, wherein the slider element comprises a passage opening connected to the guide receptacle, wherein the passage opening overlaps the notch of the coupling element, and wherein the coupling element comprises at least one outwardly projecting guide pin, on an axis parallel to the pivot axis, and the slider element comprises at least one guide gap, which runs parallel to the coupling axis and is connected to the guide receptacle, wherein the at least one guide pin is arranged in the at least one guide gap.

7. The coupling device according to claim 6, wherein the at least one guide pin is configured to cooperate with the at least one pivot bracket and protrudes with an end portion from the guide gap of the slider element, wherein the pivot bracket comprises a guide groove which runs in the radial direction to the pivot bearing device and in which the end portion of the guide pin is received.

8. The coupling device according to claim 1, wherein the coupling device comprises a spring element which exerts a force in the direction of the coupling axis on the coupling element and holds the coupling element in the engaging position.

9. The coupling device according to claim 8, wherein the spring element is arranged in a receiving recess of the slider element, which comprises a base and adjoins the guide receptacle to form a step, and/or the coupling element comprises a connecting piece facing away from an opening side of the recess for engagement with the spring element.

10. The coupling device according to claim 4, wherein the bearing component comprises a block portion on which the guide rails are arranged, wherein the block portion comprises a through-opening along the longitudinal axis for the force transmission element.

11. A handle device for a surgical instrument for arrangement at a proximal end of a shaft through which the force transmission element extends, which force transmission element defines the longitudinal axis and comprises an the offset connection element at a proximal end, wherein the handle device comprises the handle part movable about a pivot axis and the coupling device for connecting the movable handle part to the force transmission element, wherein the coupling device is the coupling device according to claim 1.

12. A surgical instrument, having a shaft through which the force transmission element extends, which is movable along a longitudinal axis and comprises the offset connection element at a proximal end, wherein the surgical instrument comprises the handle device, which comprises the handle part at a proximal end of the shaft, which handle part can move about a pivot axis running perpendicular to the longitudinal axis, and comprises a tool at a distal end of the shaft which is operatively connected to the force transmission element, and wherein the surgical instrument comprises the coupling device for connecting the movable handle part to the force transmission element, wherein the coupling device is the coupling device according to claim 1.

13. A method for assembling a surgical instrument with a handle device and a force transmission element with an offset connection element at a proximal end using a coupling device, wherein the coupling device comprises: a coupling element configured to connect the force transmission element to a movable handle part of the handle device, the coupling element comprising a recess for receiving the connection element; and a slider element which cooperates with the coupling element and is configured to cooperate with the movable handle part and to move in a direction of the longitudinal axis, wherein the coupling element is received in a guide receptacle of the slider element so as to be movable along a coupling axis which runs perpendicular to the longitudinal axis, wherein the recess in the coupling element is configured corresponding to the coupling axis and has a cross section which tapers in a direction of the coupling axis, and the coupling element comprises a notch which is connected to the recess, wherein a width of the notch tapers in a direction parallel to the coupling axis, and wherein the coupling element is configured to move along the coupling axis between at least one release position, in which at a height of the longitudinal axis the width of the notch is greater than a width of the connection element, and at least one engaging position in which, at the height of the longitudinal axis, a cross-sectional dimension of the recess in the direction of the longitudinal axis corresponds to a longitudinal section dimension of the connection element and the width of the notch is smaller than the width of the connection element, so that the connection element engages behind the notch and comes to rest in the recess, the method comprising the steps of:
arranging the coupling element in the guide receptacle of the slider element along the coupling axis in the release position, in which at the height of the longitudinal axis the width of the notch is greater than the width of the connection element;
introducing the connection element at the proximal end of the force transmission element along the longitudinal axis into the coupling device through the notch into the recess; and
transferring the coupling element into the engaging position in which, at the height of the longitudinal axis, the cross-sectional dimension of the recess in the direction of the longitudinal axis corresponds to the longitudinal section dimension of the connection element and the width of the notch is smaller than the width of the connection element, so that the connection element engages behind the notch and comes to rest in the recess.

14. The method according to claim 13, wherein the force transmission element is configured to transmit a predetermined limit force and is selected according to a maximum limit force predetermined for a tool which is arranged at a distal end of the force transmission element.

15. A method for disassembling a surgical instrument with a handle device and a force transmission element with an offset connection element at a proximal end using a coupling device, wherein the coupling device comprises: a coupling element configured to connect the force transmission element to a movable handle part of the handle device, the coupling element comprising a recess for receiving the connection element; and a slider element which cooperates with the coupling element and is configured to cooperate with the movable handle part and to move in a direction of the longitudinal axis, wherein the coupling element is received in a guide receptacle of the slider element so as to be movable along a coupling axis which runs perpendicular to the longitudinal axis, wherein the recess in the coupling element is configured corresponding to the coupling axis and has a cross section which tapers in a direction of the coupling axis, and the coupling element comprises a notch which is connected to the recess, wherein a width of the notch tapers in a direction parallel to the coupling axis, and wherein the coupling element is configured to move along the coupling axis between at least one release position, in which at a height of the longitudinal axis the width of the notch is greater than a width of the connection element, and at least one engaging position in which, at the height of the longitudinal axis, a cross-sectional dimension of the recess in the direction of the longitudinal axis corresponds to a longitudinal section dimension of the connection element and the width of the notch is smaller than the width of the connection element, so that the connection element engages behind the notch and comes to rest in the recess, the method comprising the steps of:
transferring the coupling element from the engaging position in which, at the height of the longitudinal axis, the cross-sectional dimension of the recess in the direction of the longitudinal axis corresponds to the longitudinal section dimension of the connection element and the width of the notch is smaller than the width of the connection element, to the release position in which, at the height of the longitudinal axis, the width of the notch is greater than the width of the connection element; and
removing the connection element from the recess of the coupling element through the notch by pulling the force transmission element out along the longitudinal axis.

16. The coupling device according to claim 2, wherein the recess in the coupling element is conical or frustoconical with a circular cross section and the connection element is spherical, wherein the cross-sectional dimension of the recess in the direction of the longitudinal axis is a diameter of the circular cross section and the longitudinal section dimension of the connection element is a diameter of the spherical connection element.
